(19) United States
(12) Patent Application Publication
(10) Pub. No.: US 2024/0016656 A1
(43) Pub. Date: Jan. 18, 2024

(54) CONTROL DEVICE AND CONTROL METHOD

(71) Applicant: Sony Group Corporation, Tokyo (JP)

(72) Inventor: Kentaro TERASAKI, Tokyo (JP)

(73) Assignee: Sony Group Corporation, Tokyo (JP)

(21) Appl. No.: 18/253,212

(22) PCT Filed: Nov. 15, 2021
(86) PCT No.: PCT/JP2021/041827

§ 371 Date: May 17, 2023

(30) Foreign Application Priority Data
Nov. 26, 2020 (JP) 2020-196078

(51) Int. Cl. A61F 7/00

(52) U.S. Cl. CPC A61F 7/007; A61F 2007/0093; A61F 2007/0095; A61F 2007/0086; A61F 2007/0075

(57) ABSTRACT

A control device (20) includes a control unit (23) that dynamically controls an operation of heating and cooling stimulation by a heating and cooling stimulator (30) that repeatedly gives alternate heating stimulation and cooling stimulation to a body part of a user.

### FIELD

The present disclosure relates to a control device and a control method.

### BACKGROUND

In recent years, a temperature control device that is used via being put on clothing or the like has been proposed to live more comfortably in daily life while efforts in consideration of global warming or the like such as cool bis has been made (Patent Literature 1). The temperature control device performs temperature control in accordance with a situation in which a user of the temperature control device is placed according to temperature, humidity, and the like measured by a sensor group or the like.

In addition, as a method for early recovering from physical fatigue after exercise or the like, a hot and cold contrast bathing that involves alternately taking hot baths and cold baths is known (Makoto Katahira et al., “Journal of Training Science for Exercise and Sports (1349-4414)”, Japan Society for Training Science for Exercise and Sport, September 2007, Vol. 19, No. 3, pp. 239-246). It has been confirmed that the hot and cold contrast bathing is effective in recovering from physical fatigue earlier than general body washing in a shower.

### CITATION LIST

### Patent Literature

Patent Literature 1: WO 2020/066564 A1

### SUMMARY

### Technical Problem

However, it is difficult to take the hot and cold contrast bathing readily. For example, there is a concern that two baths are required and the temperatures need to be maintained in terms of equipment. In addition, there are a hygiene concern when the baths are used by a large number of people, and an issue of adaptability for people with physical difficulty, and the like.

Therefore, the present disclosure proposes a control device and a control method capable of imitating the hot and cold contrast bathing easily.

### Solution to Problem

To solve the above problem, A control device that provides a service that requires an identity verification process according to an embodiment of the present disclosure includes: a control unit that dynamically controls an operation of heating and cooling stimulation by a heating and cooling stimulator that repeatedly gives alternate heating stimulation and cooling stimulation to a body part of a user.

### BRIEF DESCRIPTION OF DRAWINGS

FIG. 1 is a diagram illustrating an outline of information processing according to an embodiment of the present disclosure.

FIG. 2 is a diagram indicating an example of items of a stimulation parameter and adjustment variables according to the embodiment of the present disclosure.

FIG. 3 is a diagram indicating an example of variable adjustment based on an operation mode according to the embodiment of the present disclosure.

FIG. 4 is a diagram indicating an example of variable adjustment based on an attribute of a user according to the embodiment of the present disclosure.

FIG. 5 is a diagram indicating an example of variable adjustment based on a stimulated portion and a condition of the stimulated portion according to the embodiment of the present disclosure.

FIG. 6 is a diagram indicating an example of variable adjustment based on a stimulated portion and a condition of the stimulated portion according to the embodiment of the present disclosure.

FIG. 7 is a diagram illustrating a concept of an operation of heating and cooling stimulation according to the embodiment of the present disclosure.

FIG. 8 is a diagram illustrating an example of relationship between temporal transition of the heating and cooling stimulation and temporal transition of temperature of the skin surface according to the embodiment of the present disclosure.

FIG. 9 is a schematic view illustrating a configuration example of a system according to the embodiment of the present disclosure.

FIG. 10 is a block diagram illustrating a configuration example of a terminal device according to the embodiment of the present disclosure.

FIG. 11 is a block diagram illustrating a configuration example of a control device according to the embodiment of the present disclosure.

FIG. 12 is a diagram indicating an outline of information defined in a parameter determination table according to the embodiment of the present disclosure.

FIG. 13 is a diagram indicating an outline of information defined in the parameter determination table according to the embodiment of the present disclosure.

FIG. 14 is a diagram indicating an outline of information defined in the parameter determination table according to the embodiment of the present disclosure.

FIG. 15 is a block diagram illustrating a configuration example of a heating and cooling stimulator according to the embodiment of the present disclosure.

FIG. 16 is a flowchart illustrating an example of a processing procedure of the control device according to the embodiment of the present disclosure.

FIG. 17 is a block diagram illustrating a hardware configuration example of a computer capable of implementing the control device according to the embodiment and modifications of the present disclosure.

### DESCRIPTION OF EMBODIMENT

Hereinafter, an embodiment of the present disclosure will be described in detail with reference to the drawings. Note that, in the following embodiment, by denoting the same numbers or reference signs to the same parts, redundant description may be omitted. In addition, in the present description and the drawings, a plurality of components having substantially the same functional configuration may be distinguished by different numbers or reference signs following the same number or reference sign.

The present disclosure will be described according to the following item order.
1. Outline of Information Processing
2. Configuration Example of System
3. Configuration Example of Device
4. Processing Procedure Example
Modification
6. Others
7. Hardware Configuration Example
8. Conclusion

### 1. Outline of Information Processing

<1-1. Flow of Processing in Control System>

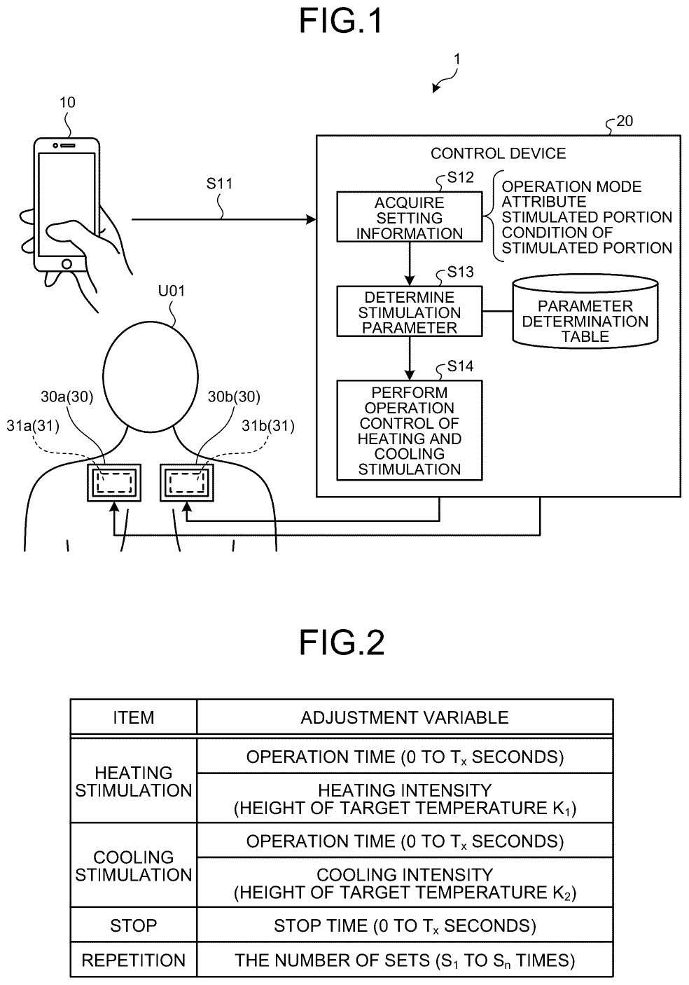

FIG. 1 is a diagram illustrating an outline of information processing according to an embodiment of the present disclosure. As illustrated in FIG. 1, a control system 1 according to the embodiment of the present disclosure includes a terminal device 10, a control device 20, and a heating and cooling stimulator 30. The terminal device 10 and the control device 20 can communicate with each other through a predetermined network. The control device 20 and the heating and cooling stimulator 30 can communicate with each other through a predetermined network. Note that FIG. 1 illustrates an example of the control system 1 according to the embodiment, and the control system 1 is not necessarily limited to the aspect illustrated in FIG. 1. For example, the control system 1 may be constituted by the control device 20 and the heating and cooling stimulator 30. In this case, a user U01 to be described below who is a user of the heating and cooling stimulator 30 or an operator of the heating and cooling stimulator 30 such as a medical worker or a service provider can directly operate the control device 20 to control the operation of the heating and cooling stimulator 30.

The terminal device 10 is a terminal device operated by the user U01, and is typically a smartphone. The user U01 may be an owner of the terminal device 10 or may be merely a user. The control device 20 is a control device that integrally controls the heating and cooling stimulator 30. The heating and cooling stimulator 30 is a device that is attached to the user U01 who is a user thereof, and performs the operation of the heating and cooling stimulation for repeatedly giving alternate heating stimulation and cooling stimulation to a body part of the user U01. The heating and cooling stimulator 30 includes a temperature change unit 31 including a semiconductor element such as a Peltier element. In the example illustrated in FIG. 1, a heating and cooling stimulator 30a and a heating and cooling stimulator 30b are the heating and cooling stimulators 30 having the same functional configuration. In the example illustrated in FIG. 1, the heating and cooling stimulator 30a is attached to the left shoulder of the user U01 with a temperature change unit 31a facing the user U01, and the heating and cooling stimulator 30b is attached to the right shoulder of the user U01 with a temperature change unit 31b facing the user U01.

In the control system 1 as described above, the terminal device 10 receives, from the user U01, setting information for operating the heating and cooling stimulator 30. Then, the terminal device 10 transmits, to the control device 20, the setting information received from the user U01 (step S11).

The control device 20 acquires the setting information transmitted from the terminal device 10 (step S12). Then, the control device 20 determines a stimulation parameter according to the setting information referring to parameter determination tables defined in advance (step S13). Then, the control device 20 transmits control signals for operating the heating and cooling stimulator 30 according to the determined stimulation parameter individually to the heating and cooling stimulator 30a and the heating and cooling stimulator 30b to perform the operation control of the heating and cooling stimulation (step S14).

<1-2. Configuration Example of Stimulation Parameter>

Items of the stimulation parameter and adjustment variables determined by the control device 20 will be described with reference to FIG. 2. FIG. 2 is a diagram indicating an example of items of the stimulation parameter and adjustment variables according to the embodiment of the present disclosure.

As indicated in FIG. 2, the stimulation parameter determined by the control device 20 is configured by associating the content of the adjustment variable with each item indicating an operation constituting the heating and cooling stimulation. The items of the operations constituting the heating and cooling stimulation include the “heating stimulation”, the “cooling stimulation”, “stop”, and “repetition”. An operation time and heating intensity are associated as adjustment variables corresponding to the “heating stimulation”. An operation time and cooling intensity are associated as adjustment variables corresponding to the “cooling stimulation”. A stop time is associated as an adjustment variable corresponding to the “stop”. The number of sets is associated as an adjustment variable corresponding to the “repetition”. The number of sets is the number of repetitions of a set of the operations: “heating stimulation”, “stop”, “cooling stimulation”, “stop”. Note that, instead of the number of sets of the heating and cooling stimulation, the entire time for performing the operation of the heating and cooling stimulation may be set. The user U01 may stop the operations constituting the heating and cooling stimulation any time via a manual operation.

The operation time as an adjustment variable indicates a time period for operation of “heating stimulation” or “cooling stimulation”. “Heating intensity” as an adjustment variable indicates a target temperature when performing the heating stimulation. “Cooling intensity” as an adjustment variable indicates a target temperature when performing the cooling stimulation. Stop time as an adjustment variable is an interval between the heating stimulation and the cooling stimulation, and indicates the time of “stop” of the heating and cooling stimulation.

The control device 20 individually determines the intensity and the operation time of the heating stimulation, the intensity and the operation time of the cooling stimulation, the stop time, and the number of sets based on the setting information acquired from the terminal device 10.

<1-3. Example of Variable Adjustment Based on Operation Mode>

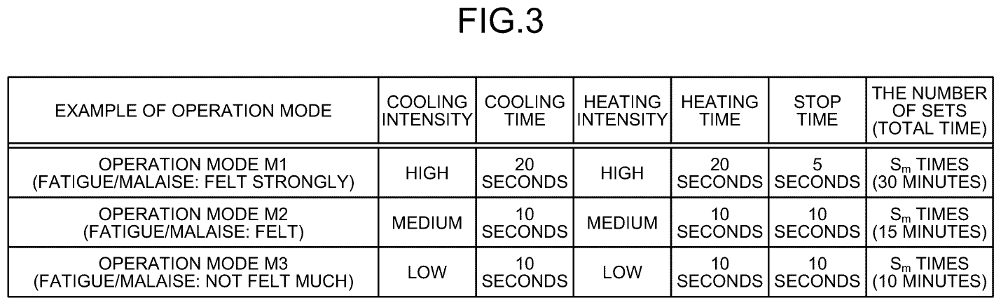

An example of variable adjustment of the stimulation parameter based on the operation mode will be described with reference to FIG. 3. FIG. 3 is a diagram indicating an example of variable adjustment based on the operation mode according to the embodiment of the present disclosure.

As indicated in FIG. 3, the control device 20 prepares in advance, for example, three types of modes of an operation mode M1, an operation mode M2, and an operation mode M3 as the operation modes for performing the heating and cooling stimulation, and determines in advance each of the setting values of the adjustment variables for each mode. The control device 20 is configured to allow the user U01 to select the operation modes M1 to M3 based on subjective fatigue/malaise. For example, the control device 20 proposes the user to select the operation mode M1 when fatigue/malaise is felt strongly, the operation mode M2 when fatigue/malaise is felt, and the operation mode M3 when fatigue/malaise is not felt much. Then, the control device 20 determines the setting values of the adjustment variables of the stimulation parameter based on the operation mode selected from these three operation modes by the user U01 of the terminal device 10.

For example, when the operation mode M1 is selected by the user U01, the control device 20 sets, with respect to the cooling stimulation, the cooling intensity, which is a setting for the target temperature of the cooling stimulation, to “high” and the cooling time, which is the operation time of the cooling stimulation, to “20 seconds”. The control device 20 sets, with respect to the heating stimulation, the heating intensity, which is a setting for the target temperature of the heating stimulation, to “high” and the heating time, which is the operation time of the heating stimulation, to “20 seconds”. With respect to the stop, the control device 20 sets the stop time to “5 seconds”. In addition, the control device sets the number of sets to “Sₘ times”. Accordingly, the control device 20 can easily adjust the operation content of the heating and cooling stimulation based on the operation mode selected by the user of the heating and cooling stimulator 30.

<1-4. Example of Variable Adjustment Based on Attribute of User>

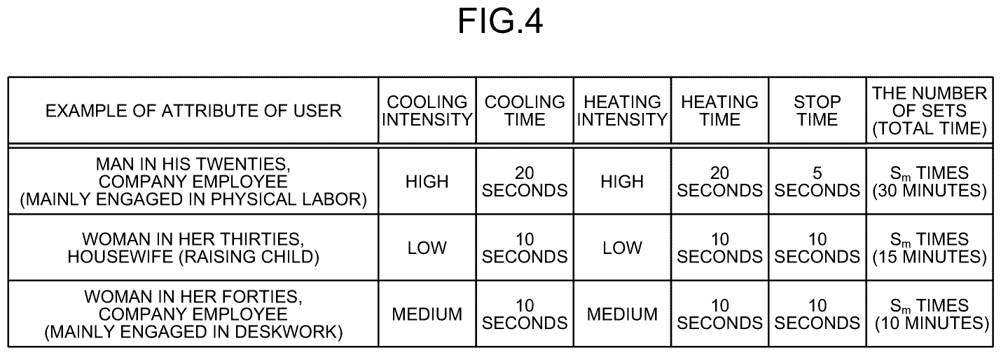

An example of variable adjustment of the stimulation parameter based on an attribute of the user will be described with reference to FIG. 4. FIG. 4 is a diagram indicating an example of variable adjustment based on the attribute of the user according to the embodiment of the present disclosure.

As indicated in FIG. 4, the control device 20 determines, in advance, setting values of the adjustment variables for each combination of age, gender, and content of work that are set in advance as the attribute of the user U01 who is the user thereof. Then, the control device determines the setting values of the adjustment variables of the stimulation parameter based on the attribute of the user U01.

For example, in a case where the user U01 of the terminal device 10 is a man in his twenties who is a company employee mainly engaged in physical labor, the control device 20 sets, with respect to the cooling stimulation, the cooling intensity, which is the target temperature setting of the cooling stimulation, to “high” and the cooling time, which is the operation time of the cooling stimulation, to “20 seconds”. The control device sets, with respect to the heating stimulation, the heating intensity, which is a setting for the target temperature of the heating stimulation, to “high” and the heating time, which is the operation time of the heating stimulation, to “20 seconds”. With respect to the stop, the control device 20 sets the stop time to “5 seconds”.

For example, in consideration of physical characteristics of women that their skin is likely to be relatively thin, the intensity of the heating and cooling stimulation in a case where the user U01 is a woman is set to be relatively lower than the intensity of the heating and cooling stimulation in a case where the user U01 is a man. In consideration of the ease and degree of accumulation of muscle fatigue depending on the content of work, the intensity of the heating and cooling stimulation in a case where the content of work of the user U01 is physical labor is set relatively higher than the intensity of the heating and cooling stimulation in a case where the content of work of the user U01 is deskwork. In addition, the variables may be adjusted based on the preference of the user U01 as the attribute of the user. For example, the preference corresponds to the subjective preference of the user U01 such as preferring stronger heating and cooling stimulation or preferring weaker heating and cooling stimulation. The control device 20 can adjust the intensity of the heating and cooling stimulation according to the preference of the user U01 by managing the use history of the user U01. In addition, the variables may be adjusted based on how the user U01 feels (sensitivity) the intensity of heating/cooling in the past treatment as the attribute of the user. For example, the control device 20 can adjust the intensity of heating and cooling stimulation according to how the user feels by acquiring and accumulating the questionnaire of the user after the treatment. Accordingly, the control device 20 can adjust the operation content of the heating and cooling stimulation such that the heating and cooling stimulation is appropriate according to the attribute of the user of the heating and cooling stimulator 30.

<1-5. Variable Adjustment Based on Stimulated Portion and Condition of Stimulated Portion>

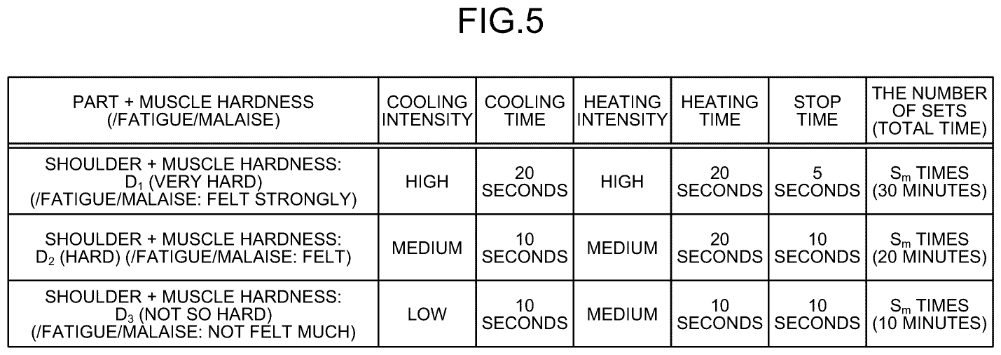

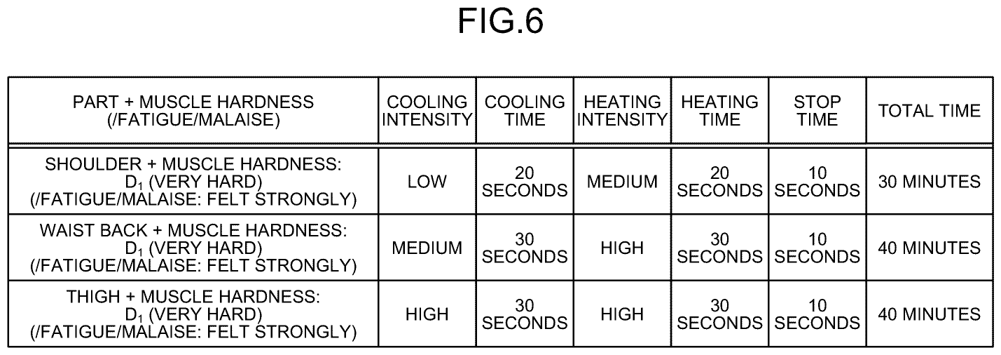

An example of variable adjustment of the stimulation parameter based on a stimulated portion and a condition of the stimulated portion will be described with reference to FIG. 5. FIGS. 5 and 6 are diagrams indicating an example of variable adjustment based on the stimulated portion and the condition of the stimulated portion according to the embodiment of the present disclosure.

For example, the control device 20 determines in advance the setting values of the adjustment variables of the stimulation parameter for each combination of the stimulated portion and the condition of the stimulated portion. The stimulated portion is a body part of the user of the heating and cooling stimulator 30, and corresponds to the neck, the waist and back, the forearm/upper arm, the thigh/shin, and the like. The condition of the stimulated portion corresponds to, for example, the muscle hardness of the muscle corresponding to the body part that is the stimulated portion, or the subjective fatigue degree (fatigue/malaise) of the user with respect to the part.

In a case where the muscle hardness can be acquired from the terminal device 10, the control device 20 acquires, from the terminal device 10, and inputs the value of the muscle hardness together with the information about the stimulated portion. In a case where the subjective fatigue degree of the user U01 can be acquired from the terminal device 10, the control device 20 has the user U01 select in advance the current fatigue degree from the choices presented to the user U01, and acquires and inputs information corresponding to the fatigue degree selected by the user U01 together with the information about the stimulated portion. Note that the muscle hardness and the fatigue degree may be directly set to the control device 20 by the user U01 or the operator of the terminal device 10 via a manual operation using the input device included in the control device 20. Then, the control device 20 determines the setting values of the adjustment variables of the stimulation parameter based on the stimulated portion and the condition of the stimulated portion specified by the user U01 of the terminal device 10.

Even if the stimulated portion is the same, the control device 20 can change the setting values of the adjustment variables of the stimulation parameter based on the condition of the stimulated portion. FIG. 5 indicates an example of the setting values of the adjustment variables in a case where, at the same stimulated portion, the conditions of the stimulated portion are different. Note that, the control device 20 can determine the setting values of the adjustment variables for each combination of the stimulated portion and the condition of the stimulated portion with respect to other parts such as the neck, the waist and back, the forearm/upper arm, and the thigh/shin that can be treated via attaching the heating and cooling stimulator 30 similarly to the example indicated in FIG. 5.

For example, even if the stimulated portion is the same, the control device 20 can determine the setting values of the adjustment variables such that the intensity of heating/cooling is stronger and the operation time is longer as the height of the muscle that is indicated by the muscle hardness is more or the fatigue/malaise of the user is more.

For example, as indicated in FIG. 5, in a case where the condition of the stimulated portion acquired from the terminal device 10 is muscle hardness: Di (very hard) or fatigue/malaise: “felt strongly”, the control device 20 can set, with respect to the cooling stimulation, the cooling intensity, which is the target temperature setting of the cooling stimulation, to “high” and the cooling time, which is the operation time of the cooling stimulation, to “20 seconds”. With respect to the heating stimulation, the control device 20 can set the heating intensity, which is a setting for the target temperature of the heating stimulation, to “high” and the heating time, which is the operation time of the heating stimulation, to “20 seconds”. The control device 20 can also set the stop time, which is the interval between the heating stimulation and the cooling stimulation, to “5 seconds”. On the other hand, in a case where the condition of the stimulated portion acquired from the terminal device 10 is muscle hardness: D₃ (not so hard) or fatigue/malaise: “not felt much”, the control device 20 can set, with respect to the heating stimulation, the cooling intensity, which is the target temperature setting of the cooling stimulation, to “low” and the cooling time, which is the operation time of the cooling stimulation, to “10 seconds”. With respect to the heating stimulation, the control device 20 sets the heating intensity, which is a setting for the target temperature of the heating stimulation, to “high” and the heating time, which is the operation time of the heating stimulation, to “10 seconds”. The control device 20 can also set the stop time, which is the interval between the heating stimulation and the cooling stimulation, to “10 seconds”.

Furthermore, even if the conditions of the stimulated portions are the same, the control device 20 can change the setting values of the adjustment variables of the stimulation parameter according to the stimulated portion. FIG. 6 indicates an example of the setting values of the adjustment variables in a case where the stimulated portions are different under the same conditions.

For example, even if the muscle hardness or the fatigue/malaise of the user is the same, the control device can determine the setting values of the adjustment variables such that the intensity of heating/cooling is stronger and the operation time is longer as the muscle tissue to be stimulated is bigger and of which skin is thicker such as the waist back and the thigh than in a case of stimulating muscle tissue that is smaller and of which skin is thinner such as the neck and the shoulder.

For example, as indicated in FIG. 6, when the stimulated portion acquired from the terminal device 10 is the shoulder, the control device 20 can set, with respect to the cooling stimulation, the cooling intensity, which is the target temperature setting of the cooling stimulation, to “low” and the cooling time, which is the operation time of the cooling stimulation, to “20 seconds”. In addition, with respect to the heating stimulation, the control device can set the heating intensity, which is the target temperature setting of the heating stimulation, to “medium” and the heating time, which is the operation time of the heating stimulation, to “20 seconds”. The control device can also set the stop time, which is the interval between the heating stimulation and the cooling stimulation, to “10 seconds”. On the other hand, when the stimulated portion acquired from the terminal device 10 is the waist back, the control device 20 can set, with respect to the cooling stimulation, the cooling intensity, which is the target temperature setting of the cooling stimulation, to “medium” and the cooling time, which is the operation time of the cooling stimulation, to “30 seconds”. With respect to the heating stimulation, the control device 20 sets the heating intensity, which is a setting for the target temperature of the heating stimulation, to “high” and the heating time, which is the operation time of the heating stimulation, to “30 seconds”. The control device can also set the stop time, which is the interval between the heating stimulation and the cooling stimulation, to “10 seconds”.

Note that, in a case where a part close to the center of the body of the user U01 is the stimulated portion, such as the waist and back, the control device 20 can adjust the cooling intensity to be lower than in a case of stimulating the thigh, which is a part away from the center of the body of the user U01, even if the muscle hardness or the fatigue/malaise is the same. Accordingly, the control device 20 can adjust the operation content of the heating and cooling stimulation such that the heating and cooling stimulation is appropriate according to the stimulated portion where the heating and cooling stimulation is performed and the condition of the stimulated portion.

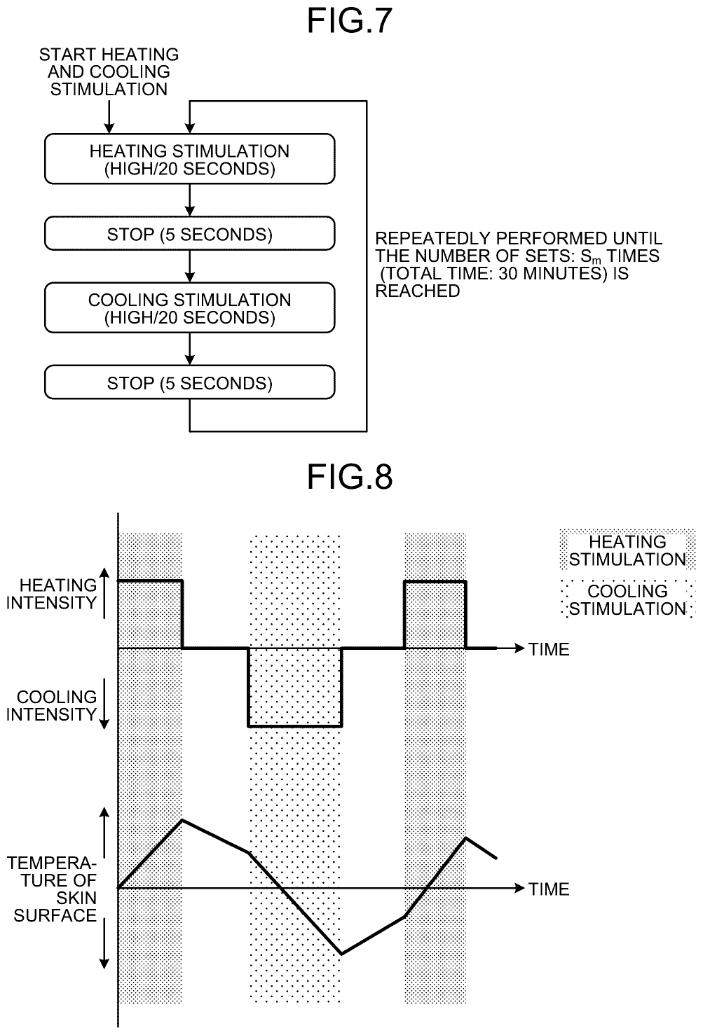

A concept of the operation of the heating and cooling stimulation will be described with reference to FIG. 7. FIG. 7 is a diagram illustrating the concept of the operation of the heating and cooling stimulation according to the embodiment of the present disclosure. For example, when the user U01 selects the operation mode M1, the control device 20 sets the cooling intensity and the time period of the stimulation parameters to “high” and “20 seconds”, sets the heating intensity and the time period to “high” and “20 seconds”, sets the stop to “5 seconds”, and sets the number of sets to “Sₘ times”. Then, the control device 20 transmits, to the heating and cooling stimulator a control signal for performing the operation of the heating and cooling stimulation in accordance with the stimulation parameter that has been set. As illustrated in FIG. 7, a set of heating stimulation (high/20 seconds)→stop (5 seconds)→cooling stimulation (high/20 seconds)→stop (5 seconds) is set as one set, and the heating and cooling stimulator 30 repeatedly performs the operation of the heating and cooling stimulation until the number of sets: Sm (times) (total time: 30 minutes) is reached.

<1-6. Relationship Between Temporal Transition of Heating and Cooling Stimulation and Temporal Transition of Temperature of Skin Surface>

The relationship between temporal transition of the heating and cooling stimulation and temporal transition of temperature of the skin surface will be described with reference to FIG. 8. FIG. 8 is a diagram illustrating an example of relationship between temporal transition of the heating and cooling stimulation and temporal transition of temperature of the skin surface according to the embodiment of the present disclosure. The upper part of FIG. 8 illustrates an example of temporal transition of the heating and cooling stimulation, and the lower part of FIG. 8 illustrates an example of temporal transition of the temperature of the skin surface of the user U01.

As illustrated in FIG. 8, upon the heating stimulation of the next cycle is then started, the temperature of the skin surface of the user U01 continues to increase at a constant rate while the heating stimulation is continued. Subsequently, when the heating stimulation is stopped, the increased temperature of the skin surface is reversed and continues to decrease at a constant rate while the stop of the heating stimulation. The cooling stimulation is then started, and while the cooling stimulation is continued, the temperature of the skin surface continues to decrease at a rate larger than the rate while the stop of the heating stimulation. Subsequently, when the cooling stimulation is stopped, the decreased temperature of the skin surface is reversed and continues to increase at a constant rate while stop of the cooling stimulation. Upon the start of the heating stimulation of the next cycle is then started, the temperature of the skin surface continues to increase again at a rate larger than the rate while the stop of the cooling stimulation. Accordingly, the control device 20 can control the operation of the heating and cooling stimulator 30 so as to repeatedly give the alternate heating and cooling stimulation to the body part of the user U01.

### 2. Configuration Example of System

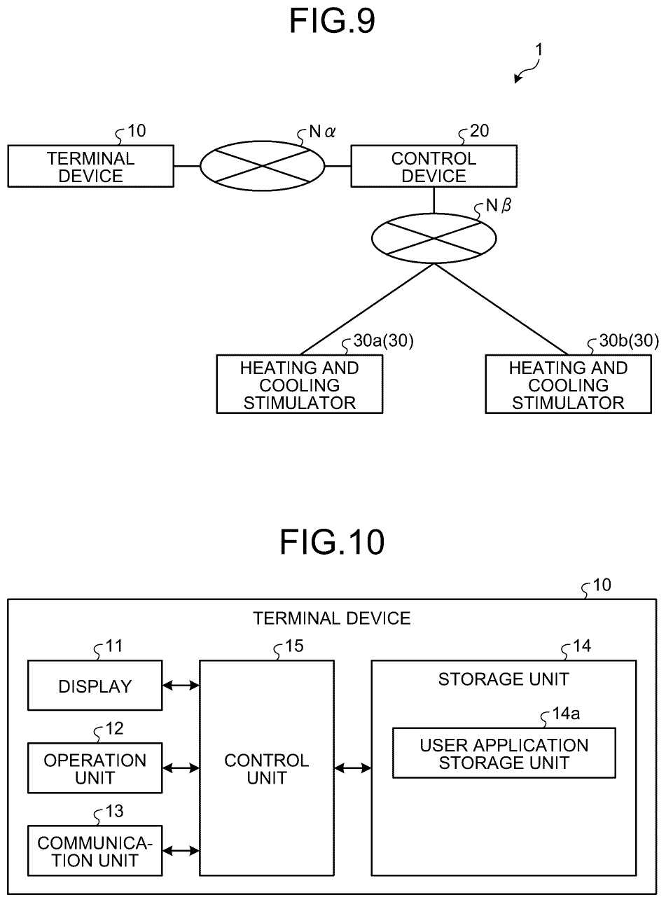

Hereinafter, a configuration example of a control system according to the embodiment of the present disclosure will be described. FIG. 9 is a schematic view illustrating a configuration example of a system according to the embodiment of the present disclosure.

As illustrated in FIG. 9, the control system 1 according to the embodiment of the present disclosure includes the terminal device 10, the control device 20, and a plurality of the heating and cooling stimulators 30. Note that the configuration of the control system 1 is not particularly limited to the example illustrated in FIG. 9, and the control system 1 may include more heating and cooling stimulators 30 than those illustrated in FIG. 9. The control system 1 may be constituted by the control device 20 and the heating and cooling stimulators 30. In this case, the setting information for operation control of the heating and cooling stimulator 30 transmitted from the terminal device 10 to the control device 20 is directly set to the control device 20 by the user U01 or the operator of the terminal device 10 via a manual operation using the input device included in the control device 20.

The terminal device 10 and the control device 20 are connected to a network Na in a wired or wireless manner. The terminal device 10 and the control device 20 can communicate with each other through the network Na. The network Nα includes a short-range wireless network such as Bluetooth (registered trademark) and ZigBee (registered trademark), a wireless local area network (LAN) such as WiFi (registered trademark), and a mobile communication network such as long term evolution (LTE) and 5G.

The control device 20, the heating and cooling stimulator 30a, and the heating and cooling stimulator 30b are wirelessly connected to a network Nβ. The control device 20 and the heating and cooling stimulator 30a can communicate each other through the network Nβ. The control device 20 and the heating and cooling stimulator 30b can communicate each other through the network Nβ. The network Nβ includes a short-range wireless network such as Bluetooth (registered trademark) and ZigBee (registered trademark), and a wireless local area network (LAN) such as WiFi (registered trademark).

Note that the terminal device 10, the control device 20, and the heating and cooling stimulator 30 may be connected to the same network.

The terminal device 10 is a terminal device operated by the user U01, and is typically a smartphone. The terminal device 10 transmits, to the control device 20, the setting information for the control device 20 to control the heating and cooling stimulator 30.

The control device 20 is a control device that integrally controls the operation of the heating and cooling stimulation by the heating and cooling stimulator 30. The control device 20 performs the operation control of the heating and cooling stimulation by the heating and cooling stimulator 30 based on the setting information acquired from the terminal device 10.

The heating and cooling stimulator 30 is a device that is attached to a body part of a user such as the user U01 of the terminal device 10, and gives the heating and cooling stimulation to the body part of the user. The heating and cooling stimulator 30 is configured to be attachable to the skin or clothes of the user.

### 3. Configuration Example of Device

<3-1. Configuration Example of Terminal Device>

Hereinafter, a configuration of the terminal device 10 according to the embodiment of the present disclosure will be described. FIG. 10 is a block diagram illustrating a configuration example of a terminal device according to the embodiment of the present disclosure.

As illustrated in FIG. 10, the terminal device 10 includes a display 11, an operation unit 12, a communication unit 13, a storage unit 14, and a control unit 15.

The display 11 displays various types of information. The display 11 can display, for example, a setting window for transmitting, to the control device 20, the setting information for controlling the operation of the heating and cooling stimulator 30. The display 11 is implemented by a display device such as an organic EL display using a liquid crystal display (LCD) or an organic light emitting diode (OLED). Note that the display 11 may be implemented by a touch panel display. In this case, the display 11 can also function as an operation unit 12 to be described below.

The operation unit 12 receives various operations. The operation unit 12 receives, for example, an input operation of the setting information for controlling the operation of the heating and cooling stimulator 30. The operation unit 12 is implemented by a button, a key, a touch panel, or the like.

The communication unit 13 transmits and receives various types of information to and from the control device via the network Na. The communication unit 13 is implemented by a communication module, a modem, or the like for communicating via a short-range wireless network, a wireless local area network (LAN), or a mobile communication network.

The storage unit 14 is implemented by, for example, a semiconductor memory element such as a random access memory (RAM) or a flash memory, or a storage device such as a hard disk or an optical disk. The storage unit 14 can store, for example, programs, data, and the like for implementing various processing functions executed by the control unit 15. The programs stored in the storage unit 14 include a program for implementing processing functions corresponding to the units of the control unit 15. The programs stored in the storage unit 14 include an operating system (OS) and various application programs.

As illustrated in FIG. 10, the storage unit 14 includes a user application storage unit 14a. A user application stored in the user application storage unit 14a provides various functions for using the heating and cooling stimulator 30. The function provided by the user application enables the user U01 of the terminal device 10 to transmit, to the control device 20, the setting information for controlling the operation of the heating and cooling stimulator 30.

The control unit 15 is implemented by a control circuit including a processor and a memory. The various processing functions executed by the control unit 15 are implemented, for example, by executing a command described in a program read from an internal memory by a processor using the internal memory as the work area. The programs read from the internal memory by the processor include an operating system (OS) and application programs. The control unit 15 may be implemented by, for example, an integrated circuit such as an application specific integrated circuit (ASIC) or a field-programmable gate array (FPGA).

The main storage device and the auxiliary storage device functioning as the above-described internal memory is implemented by, for example, a semiconductor memory element such as a random access memory (RAM) or a flash memory, or a storage device such as a hard disk or an optical disk.

The function provided by the user application stored in the user application storage unit 14a causes the control unit 15 to transmit, to the control device 20, the setting information for controlling the operation of the heating and cooling stimulator 30.

<3-2. Configuration Example of Control Device>

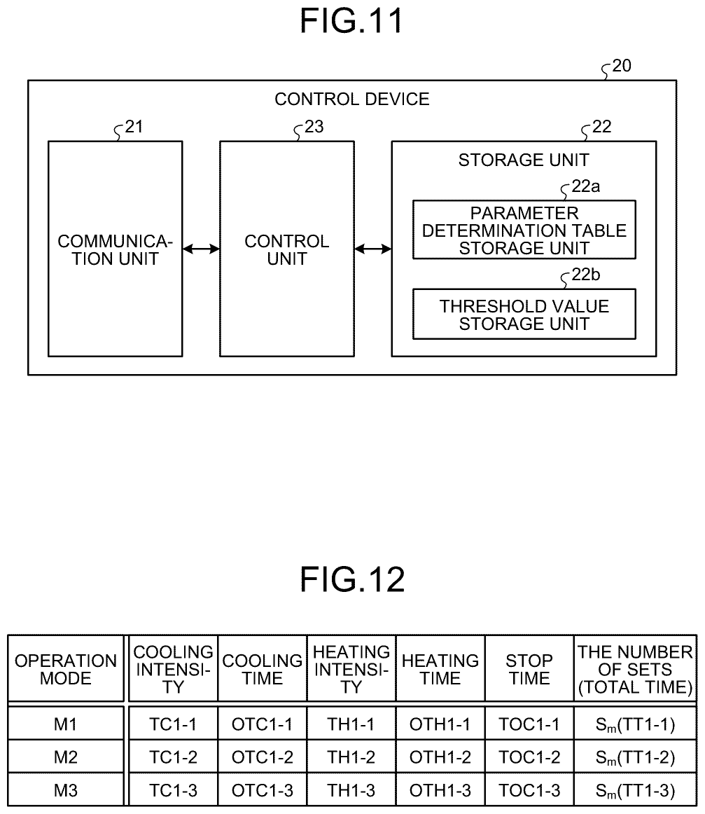

Hereinafter, a configuration of the control device 20 according to the embodiment of the present disclosure will be described with reference to FIG. 11. FIG. 11 is a block diagram illustrating a configuration example of a control device according to the embodiment of the present disclosure.

As illustrated in FIG. 11, the control device 20 includes a communication unit 21, a storage unit 22, and a control unit 23.

The communication unit 21 transmits and receives various types of information to and from the terminal device 10 via the network Nα. The communication unit 21 receives the setting information from the terminal device via the network Nα. In addition, the communication unit 21 communicates with the heating and cooling stimulator 30 via the network Nβ. The communication unit 21 transmits a control signal to the heating and cooling stimulator 30. The communication unit 21 receives information about the temperature of the heating and cooling stimulation from the heating and cooling stimulator 30. The communication unit 21 is implemented by a communication module, a modem, or the like for communicating via a short-range wireless network, a wireless local area network (LAN), or a mobile communication network.

The storage unit 22 is implemented by, for example, a semiconductor memory element such as a random access memory (RAM) or a flash memory, or a storage device such as a hard disk or an optical disk. The storage unit 22 can store, for example, programs, data, and the like for implementing various processing functions executed by the control unit 23. The programs stored in the storage unit 22 include a program of the control unit 23 for implementing processing functions corresponding to respective units. The programs stored in the storage unit 22 include an operating system (OS) and various application programs.

As illustrated in FIG. 11, the storage unit 22 includes a parameter determination table storage unit 22a and a threshold value storage unit 22b.

The parameter determination table storage unit 22a stores a parameter determination table. In the parameter determination table, as described above, information for determining the stimulation parameter for controlling the operation of the heating and cooling stimulation is defined in advance based on the setting information acquired from the terminal device 10. FIGS. 12 to 14 are diagrams indicating an outline of information defined in the parameter determination table according to the embodiment of the present disclosure.

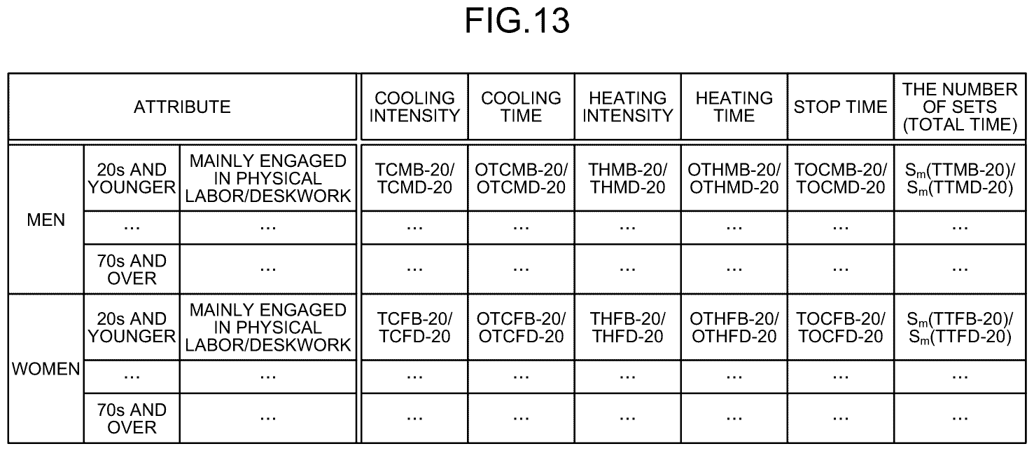

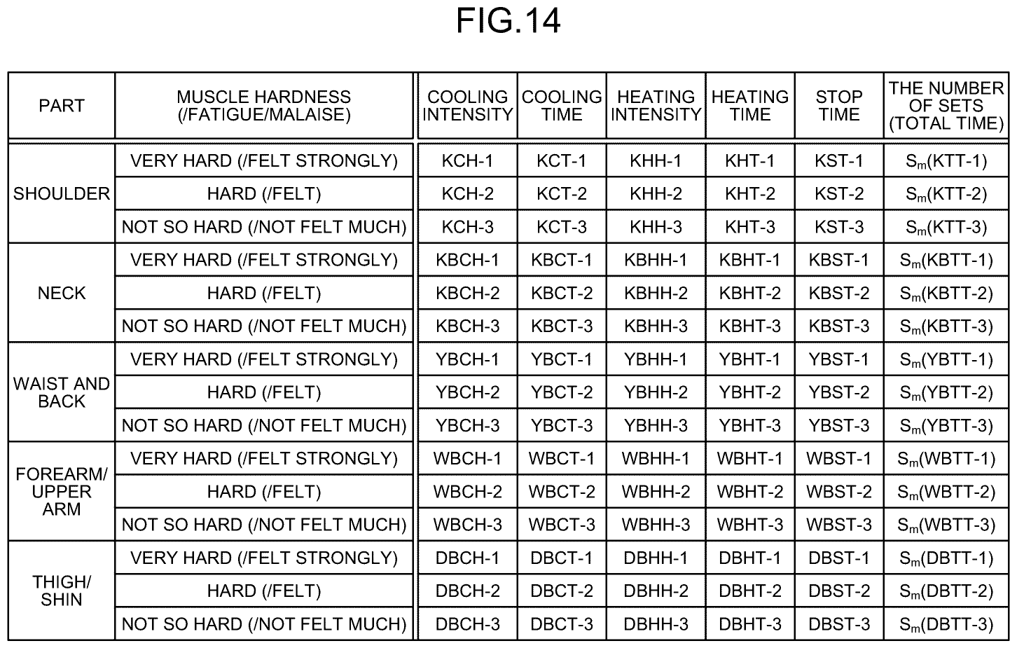

The parameter determination table indicated in FIG. 12 indicates an example in which the setting values of the adjustment variables constituting the stimulation parameter are defined for each operation mode. The parameter determination table indicated in FIG. 13 indicates an example in which the setting values of the adjustment variables constituting the stimulation parameter are defined for each attribute of the user. The parameter determination table indicated in FIG. 14 indicates an example in which the setting values of the adjustment variables constituting the stimulation parameter are defined for each stimulated portion (a body part of the user) and the condition of the stimulated portion. FIGS. 12 to 14 indicate an outline of information defined in the parameter determination table as an example, and information different from that in FIGS. 12 to 14 may be defined. For example, as the attribute of the user defined in the parameter determination table indicated in FIG. 13, the setting values of the adjustment variables may be defined according to the subjective preference (taste) of the user such as preferring stronger heating and cooling stimulation or preferring weaker heating and cooling stimulation.

A threshold value storage unit 23b stores threshold value information for determining whether the temperature information acquired from the heating and cooling stimulator 30 is within a safe range for a user receiving the heating and cooling stimulation. In the threshold value information, a threshold value for cooling intensity for determining the safety of the cooling intensity (target temperature) and a threshold value for heating intensity for determining the heating intensity (target temperature) may be stored. Furthermore, information about a safe temperature range may be stored as the threshold value information.

The control unit 23 is implemented by a control circuit including a processor and a memory. The various processing functions executed by the control unit 23 are implemented, for example, by executing a command described in a program read from an internal memory by a processor using the internal memory as the work area. The programs read from the internal memory by the processor include an operating system (OS) and application programs. The control unit 23 may be implemented by, for example, an integrated circuit such as an application specific integrated circuit (ASIC) or a field-programmable gate array (FPGA).

The main storage device and the auxiliary storage device functioning as the above-described internal memory is implemented by, for example, a semiconductor memory element such as a random access memory (RAM) or a flash memory, or a storage device such as a hard disk or an optical disk.

The control unit 23 determines the setting values of the stimulation parameter according to the setting information acquired from the terminal device 10 referring to the parameter determination table stored in a parameter determination table storage unit 23a. The control unit 23 transmits, to the heating and cooling stimulator 30, a control signal for performing the operation of the heating and cooling stimulation in accordance with the setting values of the stimulation parameter that have been determined. The control signal is a signal for determining a control current for controlling driving the Peltier element included in the heating and cooling stimulator 30.

The control unit 23 determines whether to stop the operation of the heating and cooling stimulation based on the information about the temperature of the heating and cooling stimulation received from the heating and cooling stimulator 30. Specifically, in a case where the cooling intensity of the heating and cooling stimulation is equal to or lower than the threshold value for cooling intensity, or in a case where the heating intensity of the heating and cooling stimulation is equal to or higher than the threshold value for heating intensity, the control unit 23 transmits, to the heating and cooling stimulator 30, a control signal for stopping the operation of the heating and cooling stimulation. This can prevent the user receiving the heating and cooling stimulation from being negatively affected due to the overheating or the overcooling.

<3-3. Configuration of Heating and Cooling Stimulator>

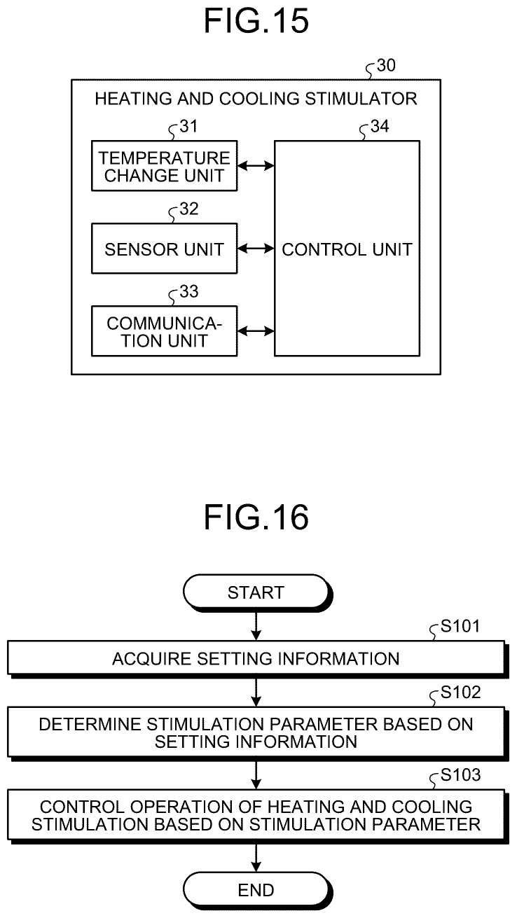

Hereinafter, a configuration of the heating and cooling stimulator 30 according to the embodiment of the present disclosure will be described with reference to FIG. FIG. 15 is a block diagram illustrating a configuration example of a heating and cooling stimulator according to the embodiment of the present disclosure.

As illustrated in FIG. 15, the heating and cooling stimulator 30 includes the temperature change unit 31, a sensor unit 32, a communication unit 33, and a control unit 34.

The temperature change unit 31 absorbs heat (cooling) or generates heat (heating) according to the direction of the control current applied from the control unit 34. The temperature change unit 31 is attached to a body part of a user of the heating and cooling stimulator (for example, the user U01 of the terminal device 10). The temperature change unit 31 can give the heating stimulation or the cooling stimulation to the body part of the user in contact with the temperature change unit 31. The temperature change unit 31 is implemented by a semiconductor element such as a Peltier element.

The sensor unit 32 detects the temperature of the heating and cooling stimulation by the temperature change unit 31 in conjunction with driving the temperature change unit 31. The sensor unit 32 transmits, to the control unit 34, the information about the temperature of the heating and cooling stimulation that has been detected.

The communication unit 33 communicates with the control device 20 via the network Nβ. The communication unit 33 receives a control signal from the control device via the network Nβ. In addition, the communication unit 33 transmits, to the control device 20, the information about the temperature of the heating and cooling stimulation via the network Nβ. The communication unit 33 is implemented by a communication module, a modem, or the like for communicating via a short-range wireless network, a wireless local area network (LAN), or a mobile communication network.

The control unit 34 is implemented by a control circuit including a processor and a memory. Various processing functions executed by the control unit 34 are implemented, for example, by executing a command described in a program read from an internal memory by a processor using the internal memory as the work area. The programs read from the internal memory by the processor include an operating system (OS) and application programs. The control unit 34 may be implemented by, for example, an integrated circuit such as an application specific integrated circuit (ASIC) or a field-programmable gate array (FPGA).

The main storage device and the auxiliary storage device functioning as the above-described internal memory is implemented by, for example, a semiconductor memory element such as a random access memory (RAM) or a flash memory, or a storage device such as a hard disk or an optical disk.

The control unit 34 applies the control current for driving the temperature change unit 31 to the temperature change unit 31 in accordance with the control signal received from the control device 20. The control unit 34 acquires information about the temperature of the heating stimulation from the sensor unit 32 in conjunction with driving the temperature change unit 31, and transmits the acquired information about the temperature to the communication unit 33.

### 4. Processing Procedure Example

Hereinafter, a processing procedure of the control device 20 according to the embodiment of the present disclosure will be described with reference to FIG. 16. FIG. 16 is a flowchart illustrating an example of the processing procedure of a control device according to the embodiment of the present disclosure. The processing procedure illustrated in FIG. 16 is performed by the control unit 23 included in the control device 20.

As illustrated in FIG. 16, the control unit 23 acquires, from the terminal device 10, the setting information for controlling the operation of the heating and cooling stimulation by the heating and cooling stimulator 30 (step S101).

Then, the control unit 23 determines the stimulation parameter (setting values) based on the setting information that has been acquired (step S102). Specifically, the control unit 23 determines the stimulation parameter according to the setting information acquired from the terminal device 10 referring to the parameter determination table stored in the parameter determination table storage unit 23a.

Then, the control unit 23 controls the operation of the heating and cooling stimulation based on the stimulation parameter that has been determined (step S103). Specifically, the control unit 23 transmits, to the heating and cooling stimulator 30, a control signal for performing the operation of the heating and cooling stimulation in accordance with the setting values of the stimulation parameter that have been determined. Then, the control unit 23 ends the processing illustrated in FIG. 16.

### 5. Modification

<5-1. Variable Adjustment Based on Environmental Temperature>

In the above embodiment, the control device 20 may adjust the intensity and the operation time of the heating stimulation, the intensity and the operation time of the cooling stimulation, and the stop time based on the environmental temperature (for example, room temperature) in the environment where the operation of the heating and cooling stimulation is performed. Specifically, the control unit 23 acquires the temperature in the room where the operation of the heating and cooling stimulation is performed. The control unit 23 may directly acquire the room temperature by the control device 20 that is provided with a function of measuring the room temperature, or may acquire the room temperature from the heating and cooling stimulator 30 that is provided with a function of measuring the room temperature. For example, in a case where the room temperature is lower than a predetermined threshold value, the control unit 23 adjusts the heating intensity to be higher and the cooling temperature to be lower. This can achieve the appropriate heating and cooling stimulation according to the environment where the heating and cooling stimulation is performed.

<5-2. Simultaneous Stimulation to Multiple Parts>

In the above embodiment, the control device 20 can control the individual heating and cooling stimulators 30 attached to the different body parts of the user in parallel, and simultaneously perform the heating and cooling stimulation to the different body parts of the user in parallel. At this time, the user attaches one heating and cooling stimulator 30 to each stimulated portion (body part) where the heating and cooling stimulation is to be performed. In addition, every time one heating and cooling stimulator is attached, the user transmits, from the terminal device 10 to the control device 20, identification information individually assigned to the heating and cooling stimulators and the setting information regarding a stimulated portion and the like. Based on the setting information received from the terminal device 10, the control device 20 recognizes the heating and cooling stimulators 30, and the association with the portion in which the heating and cooling stimulator 30 is attached. Then, the control device 20 adjusts the setting values of the stimulation parameter in accordance with the stimulated portion (body part) and the condition of the stimulated portion (muscle hardness, fatigue/malaise, and the like), and controls the heating and cooling stimulation in parallel in accordance with the stimulated portions (body part) and the conditions of the stimulated portions (muscle hardness, fatigue/malaise, and the like).

### 6. Others

The control device 20 according to the embodiment and the modifications of the present disclosure may be implemented by a dedicated computer system or a general-purpose computer system.

A control program for implementing the control method performed by the control device 20 according to the embodiment and the modifications of the present disclosure may be stored in a computer-readable recording medium such as an optical disk, a semiconductor memory, a magnetic tape, or a flexible disk and distributed. At this time, the control device 20 according to the embodiment and the modifications of the present disclosure can implement an information processing method according to the embodiment and the modifications of the present disclosure by installing various programs in a computer and executing them.

The various programs for implementing a control method executed by the control device 20 according to the embodiment and the modifications of the present disclosure may be stored in a disk device included in a server on a network such as the Internet and may be downloaded to a computer. Functions provided by the various programs for implementing the control method executed by the control device 20 according to the embodiment and the modifications of the present disclosure may be implemented in cooperation of the OS and an application program. In this case, a part other than the OS may be stored in a medium and distributed, or a part other than the OS may be stored in an application server to allow download or the like of the part to a computer. At least a part of the processing functions for implementing the control method executed by the control device 20 according to the embodiment and the modifications of the present disclosure may be implemented by a cloud server on a network. For example, when the processing functions for determining the stimulation parameter is executed on the cloud server, the terminal device 10 or the control device 20 transmits the setting information for operating the heating and cooling stimulator 30 to the cloud server. The cloud server determines the stimulation parameter based on the setting information, and returns the stimulation parameter to the terminal device 10 or the control device 20. The terminal device 10 or the control device 20 controls the operation of the heating and cooling stimulator 30 based on the stimulation parameter received from the cloud server.

Among the processing described in the embodiment and the modifications of the present disclosure, all or part of the processing described as being performed automatically can be performed manually, or all or part of the processing described as being performed manually can be performed automatically by known methods. In addition, processing procedures, specific names, and information including various types of data and parameters described and indicated in the above description and drawings may be changed in any manner unless otherwise specified. For example, the various types of information indicated in the drawings is not limited to the indicated information.

In addition, each component of the control system 1 according to the embodiment and the modifications of the present disclosure is functionally conceptual, and is not necessarily required to be configured as illustrated in the drawings. For example, in the above embodiment and modifications, the example in which the control device 20 integrally controls the heating and cooling stimulators 30 has been described. However, the processing functions for implementing the control of the control device 20 may be provided in any of the heating and cooling stimulators 30, and the heating and cooling stimulator 30 may perform the control of the control device 20. In addition, the functions for implementing the control of the control device 20 may be included in an application to be distributed to the terminal device 10, and the terminal device 10 may perform the control of the control device. Each component of the control device 20 according to the embodiment and the modifications of the present disclosure is functionally conceptual, and is not necessarily required to be physically configured as illustrated in the drawings. That is, a specific form of distribution/integration of the devices is not limited to the illustrated examples, and the devices can be configured by functionally or physically distributing/integrating all or a part thereof in any unit in accordance with various loads, usage conditions, and the like. For example, the function for the control device 20 to stop the operation of the heating and cooling stimulation based on the temperature of the heating and cooling stimulation may be distributed to the heating and cooling stimulator 30, and the heating and cooling stimulator 30 may stop the operation of the heating and cooling stimulation.

The embodiment and the modifications can be appropriately combined within the scope not causing contradiction in processing details. The order of the steps illustrated in the flowcharts according to the embodiment of the present disclosure can be changed as appropriate.

Although the embodiment and the modifications of the present disclosure have been described above, the technical scope of the present disclosure is not limited to the above-described embodiment and the modifications, and various modifications can be made without departing from the gist of the present disclosure. In addition, the components of the embodiment and the modifications may be appropriately combined.

### 7. Hardware Configuration Example

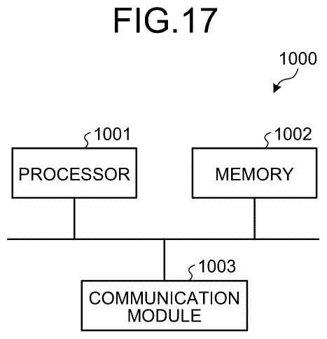

A hardware configuration example of a computer capable of implementing the control device 20 according to the embodiment and the modifications of the present disclosure will be described with reference to FIG. 17. FIG. 17 is a block diagram illustrating a hardware configuration example of a computer capable of implementing the control device according to the embodiment and the modifications of the present disclosure. Note that FIG. 17 illustrates an example of a computer, and a computer is not necessarily limited to the configuration illustrated in FIG. 17.

As illustrated in FIG. 17, the control device 20 according to the embodiment and the modifications of the present disclosure can be implemented by, for example, a computer 1000 including a processor 1001, a memory 1002, and a communication module 1003.

The processor 1001 is typically a central processing unit (CPU), a digital signal processor (DSP), a system-on-a-chip (SoC), a system large scale integration (LSI), or the like.

The memory 1002 is typically a nonvolatile or volatile semiconductor memory such as a random access memory (RAM), a read only memory (ROM), or a flash memory, a magnetic disk, or the like. The storage unit 22 included in the control device 20 is implemented by the memory 1002.

The communication module 1003 is typically a communication card for a wired or wireless local area network (LAN), long term evolution (LTE), Bluetooth (registered trademark), or wireless USB (WUSB), a router for optical communication, various communication modems, or the like. The functions of the communication unit 21 included in the control device 20 according to the above embodiment is implemented by the communication module 1003.

The processor 1001 functions as, for example, an arithmetic processing device or a control device, and controls the overall operation of the components or a part thereof based on various programs recorded in the memory 1002. The processing functions of the control unit 23 included in the control device 20 are implemented by the processor 1001 reading, from the memory 1002, a control program in which a command for operating as the control unit 23 is described and executing the control program.

That is, the processor 1001 and the memory 1002 implement the control by the control unit 23 included in the control device 20 in cooperation with software (a control program stored in the memory 1002).

### 8. Conclusion

The control device 20 according to the embodiment of the present disclosure includes the control unit 23. The control unit 23 dynamically controls the operation of the heating and cooling stimulation by the heating and cooling stimulator 30 that repeatedly gives the alternate heating stimulation and cooling stimulation to the body part of the user. Thus, the control device 20 can reproduce the hot and cold contrast bathing easily.

The heating and cooling stimulation includes a stop operation. Then, the control unit 23 determines, as the stimulation parameter that is variables for controlling the operation of the heating and cooling stimulation, the intensity and the operation time of the heating stimulation, the intensity and the operation time of the cooling stimulation, and the stop time. Accordingly, the control device 20 can flexibly configure the operation of the heating and cooling stimulation.

The control unit 23 adjusts the intensity and the operation time of the heating stimulation, the intensity and the operation time of the cooling stimulation, and the stop time based on the operation mode selected by the user among the operation modes defined in advance. Accordingly, the control device 20 can easily adjust the operation content of the heating and cooling stimulation based on the operation mode selected by the user of the heating and cooling stimulator 30.

The control unit 23 adjusts the intensity and the operation time of the heating stimulation, the intensity and the operation time of the cooling stimulation, and the stop time based on the attribute of the user. Accordingly, the control device 20 can adjust the operation content of the heating and cooling stimulation such that the heating and cooling stimulation is appropriate according to the attribute of the user of the heating and cooling stimulator 30.

The above-described attribute of the user includes sex, age, content of work, and preference of the user. Accordingly, the control device 20 can adjust the operation content of the heating and cooling stimulation according to sex, age, content of work, and preference of the user.

The control unit 23 adjusts the intensity and the operation time of the heating stimulation, the intensity and the operation time of the cooling stimulation, and the stop time based on the part of the user to which the heating and cooling stimulator 30 is attached and the condition of the part. Accordingly, the control device 20 can adjust the operation content of the heating and cooling stimulation such that the heating and cooling stimulation is appropriate according to the stimulated portion where the heating and cooling stimulation is performed and the condition of the stimulated portion.

The above-described condition of the part (stimulated portion) includes the muscle hardness or the subjective fatigue degree of the user with respect to the part. Accordingly, the control device 20 can adjust the operation content of the heating and cooling stimulation such that the heating and cooling stimulation is appropriate according to the muscle hardness or the fatigue degree of the user with respect to the part (stimulated portion).

The control unit 23 adjusts the intensity and the operation time of the heating stimulation, the intensity and the operation time of the cooling stimulation, and the stop time based on the environmental temperature in the environment where the operation of the heating and cooling stimulation is performed. Accordingly, the control device can adjust the operation content of the heating and cooling stimulation such that the heating and cooling stimulation is appropriate according to the temperature in the environment where the heating and cooling stimulation is performed.

The control unit 23 acquires, from the heating and cooling stimulator 30, the temperature of the heating and cooling stimulation while performing the operation of the heating and cooling stimulation, and when the acquired temperature exceeds the threshold value, the control unit transmits, to the heating and cooling stimulator 30, a control signal for stopping the operation of the heating and cooling stimulation. This can prevent the user receiving the heating and cooling stimulation from being negatively affected due to the overheating or the overcooling.

The heating and cooling stimulator 30 is configured using a Peltier element. Accordingly, the control device 20 can perform the heating and cooling control with a simple configuration.

The effects described in this description are merely illustrative or exemplary, and are not restrictive. That is, in addition to or in the place of the above effects, the technique according to the present disclosure may achieve other effects that are obvious to those skilled in the art from the description in this description.

Note that the following configurations also belong to the technical scope of the present disclosure.

(1)

A control device comprising:
a control unit that dynamically controls an operation of heating and cooling stimulation by a heating and cooling stimulator that repeatedly gives alternate heating stimulation and cooling stimulation to a body part of a user.

(2)

The control device according to (1), wherein
the heating and cooling stimulation includes a stop operation, and
the control unit
determines, as variables for controlling the operation of the heating and cooling stimulation, intensity and an operation time of heating stimulation, intensity and an operation time of cooling stimulation, and a stop time.

(3)

The control device according to (2), wherein
the control unit
adjusts intensity and an operation time of heating stimulation, intensity and an operation time of cooling stimulation, and a stop time based on an operation mode selected by the user among operation modes defined in advance.

(4)

The control device according to (2), wherein
the control unit
adjusts intensity and an operation time of heating stimulation, intensity and an operation time of cooling stimulation, and a stop time based on an attribute of the user.

(5)

The control device according to (4), wherein
the attribute of the user includes sex, age, content of work, and preference of the user.

(6)

The control device according to (2), wherein
the control unit
adjusts intensity and an operation time of heating stimulation, intensity and an operation time of cooling stimulation, and a stop time based on the part of the user to which the heating and cooling stimulator is attached and a condition of the part.

(7)

The control device according to (6), wherein
the condition of the part includes muscle hardness or a subjective fatigue degree of the user with respect to the part.

(8)

The control device according to (2), wherein
the control unit
adjusts intensity and an operation time of heating stimulation, intensity and an operation time of cooling stimulation, and a stop time based on an environmental temperature in environment where the operation of the heating and cooling stimulation is performed.

(9)

The control device according to (1), wherein
the control unit
acquires, from the heating and cooling stimulator, a temperature of the heating and cooling stimulation while performing the operation of the heating and cooling stimulation, and when the acquired temperature exceeds a threshold value, the control unit transmits, to the heating and cooling stimulator, a control signal for stopping the operation of the heating and cooling stimulation.

(10)

The control device according to (1), wherein
the heating and cooling stimulator is configured using a Peltier element, and
the control unit
transmits, to the heating and cooling stimulator, a control signal for controlling the operation of the heating and cooling stimulation by a Peltier element.

(11)

A control method performed by a processor, comprising:
controlling dynamically an operation of heating and cooling stimulation by a heating and cooling stimulator that repeatedly gives alternate heating stimulation and cooling stimulation to a body part of a user.

### REFERENCE SIGNS LIST

1 CONTROL SYSTEM
10 TERMINAL DEVICE
11 DISPLAY
12 OPERATION UNIT
13 COMMUNICATION UNIT
14 STORAGE UNIT
14A USER APPLICATION STORAGE UNIT
15 CONTROL UNIT
20 CONTROL DEVICE
21 COMMUNICATION UNIT
22 STORAGE UNIT
22A PARAMETER DETERMINATION TABLE STORAGE UNIT
22B THRESHOLD VALUE STORAGE UNIT
23 CONTROL UNIT
30 HEATING AND COOLING STIMULATOR
31 TEMPERATURE CHANGE UNIT
32 SENSOR UNIT
33 COMMUNICATION UNIT
34 CONTROL UNIT

1. A control device comprising:
a control unit that dynamically controls an operation of heating and cooling stimulation by a heating and cooling stimulator that repeatedly gives alternate heating stimulation and cooling stimulation to a body part of a user.

2. The control device according to claim 1, wherein
the heating and cooling stimulation includes a stop operation, and
the control unit
determines, as variables for controlling the operation of the heating and cooling stimulation, intensity and an operation time of heating stimulation, intensity and an operation time of cooling stimulation, and a stop time.

3. The control device according to claim 2, wherein
the control unit
adjusts intensity and an operation time of heating stimulation, intensity and an operation time of cooling stimulation, and a stop time based on an operation mode selected by the user among operation modes defined in advance.

4. The control device according to claim 2, wherein
the control unit
adjusts intensity and an operation time of heating stimulation, intensity and an operation time of cooling stimulation, and a stop time based on an attribute of the user.

5. The control device according to claim 4, wherein
the attribute of the user includes sex, age, content of work, and preference of the user.

6. The control device according to claim 2, wherein
the control unit
adjusts intensity and an operation time of heating stimulation, intensity and an operation time of cooling stimulation, and a stop time based on the part of the user to which the heating and cooling stimulator is attached and a condition of the part.

7. The control device according to claim 6, wherein
the condition of the part includes muscle hardness or a subjective fatigue degree of the user with respect to the part.

8. The control device according to claim 2, wherein
the control unit
adjusts intensity and an operation time of heating stimulation, intensity and an operation time of cooling stimulation, and a stop time based on an environmental temperature in environment where the operation of the heating and cooling stimulation is performed.

9. The control device according to claim 1, wherein
the control unit
acquires, from the heating and cooling stimulator, a temperature of the heating and cooling stimulation while performing the operation of the heating and cooling stimulation, and when the acquired temperature exceeds a threshold value, the control unit transmits, to the heating and cooling stimulator, a control signal for stopping the operation of the heating and cooling stimulation.

10. The control device according to claim 1, wherein
the heating and cooling stimulator is configured using a Peltier element, and
the control unit
transmits, to the heating and cooling stimulator, a control signal for controlling the operation of the heating and cooling stimulation by a Peltier element.

11. A control method performed by a processor, comprising:
controlling dynamically an operation of heating and cooling stimulation by a heating and cooling stimulator that repeatedly gives alternate heating stimulation and cooling stimulation to a body part of a user.
